(19) United States
(12) Patent Application Publication
(10) Pub. No.: US 2021/0052641 A1
(43) Pub. Date: Feb. 25, 2021

(54) PRE-CONDITIONED EXTRACELLULAR VESICLES AND METHODS OF PRODUCTION

(71) Applicant: United Therapeutics Corporation, Silver Spring, MD (US)

(72) Inventors: Pamela Tiet, Newbury Park, CA (US); Roger Ilagan, Durham, NC (US)

(73) Assignee: United Therapeutics Corporation, Silver Spring, MD (US)

(21) Appl. No.: 16/999,763

(22) Filed: Aug. 21, 2020

Related U.S. Application Data
(60) Provisional application No. 62/890,843, filed on Aug. 23, 2019.

(51) Int. Cl. A61K 35/12; C12N 5/0775; C12N 5/00

(52) U.S. Cl. CPC A61K 35/12; C12N 5/0665; C12N 5/0031; A61K 2035/124; C12N 2501/24; C12N 2513/00; C12N 2501/2306

(57) ABSTRACT

A method for treating inflammatory diseases is provided. The method may include administering to a subject in need thereof a therapeutically effective amount of isolated extracellular vesicles or exosomes obtained from mesenchymal stem cells pre-conditioned with at least TNF-α. The isolated extracellular vesicles or exosomes may exhibit (a) enhanced expression of flotillin-1, (b) enhanced expression of CD73, or (c) enhanced expression of IDO. A method of isolating extracellular vesicles or exosomes capable of treating an inflammatory disease is also provided, wherein the method may include (a) culturing stem cells in a growth medium, (b) then culturing the stem cells in a starve medium supplemented with at least TNF-α, and (c) separating exosomes or extracellular vesicles from the culture. Cultures suitable for treatment are also provided.

### CROSS-REFERENCE TO RELATED APPLICATIONS

This application claims priority to U.S. Provisional Application 62/890,843, filed Aug. 23, 2019.

### FIELD OF USE

The present application relates generally to methods of expanding and isolating extracellular vesicles, including exosomes, and the use of extracellular vesicles or exosomes in treatment of inflammatory diseases, and conditions and media associated with decreasing inflammation.

### BACKGROUND

Mesenchymal stem cells (MSCs) are a heterogeneous fibroblast-like population of cells that can be isolated from many human tissues, including, but not limited to, bone marrow, adipose, skeletal muscle, heart, umbilical cord, and placenta. MSCs have attracted the attention of scientists and clinicians due to their differentiation potential and active participation in tissue repair and regeneration after migration to the site of tissue injury. When stimulated by appropriate signals, MSCs are capable of differentiating into a number of specialized cell types such as adipocytes, osteoblasts, chondrocytes, and, less frequently, endothelial cells and cardiomyocytes. MSCs are also amenable to allogeneic transplantation and are immunoprivileged, providing greater acceptance in vivo.

In addition, MSCs possess strong immunosuppressive and immunomodulatory properties that are mediated by both cell-cell contact and production of various signaling factors. Mesenchymal stem cells (MSCs) are able to migrate towards sites of inflammation/injury by sensing inflammatory cytokines (paracrine effect). These cells are able to immunomodulate the inflammatory environment by releasing anti-inflammatory soluble factors as well as extracellular vesicles (EVs), including exosomes.

However, despite the benefits of MSCs and EVs in preclinical experimental settings, their effect in treating patients with various immune system related diseases has so far shown mixed results. This is possibly attributable to the inability of MSCs to survive and/or to function properly in an inhospitable host environment. Thus, there is a need to improve the ability of MSCs to produce EVs in a harsh microenvironment and to enhance their regulation of the immune responses by preconditioning the cells in designed or engineered environments with specific physical or chemical parameters and factors.

### SUMMARY

The present disclosure overcomes the drawbacks of previously known methods by providing a method of treating an inflammatory disease. The methods may include administering to a subject in need thereof a therapeutically effective amount of isolated extracellular vesicles or exosomes obtained from mesenchymal stem cells pre-conditioned with at least TNF-α, further wherein the isolated extracellular vesicles or exosomes have one or more of the following characteristics: (a) enhanced expression of flotillin-1, (b) enhanced expression of CD73, or (c) enhanced expression of IDO. The mesenchymal stem cells may be derived from bone marrow. In some embodiments, the mesenchymal stem cells may also be further pre-conditioned with IFN-γ or IL-6.

The inflammatory disease selected for treatment may include, but is not limited to, bronchopulmonary dysplasia, idiopathic pulmonary fibrosis, and pulmonary hypertension.

A method of isolating extracellular vesicles or exosomes capable of treating an inflammatory disease is also provided herein. Cytokine-treated MSCs cultured in two and three dimensional cultures can secrete EVs with increased immunosuppressive and angiogenic activity. The method may include, but is not limited to, the following steps: (a) culturing stem cells in a growth medium, (b) then culturing the stem cells in a starve medium supplemented with at least TNF-α, and (c) separating exosomes or extracellular vesicles from the culture. In some embodiments, the stem cells of the method may be mesenchymal stem cells. The mesenchymal stem cells may be derived from bone marrow.

In certain embodiments, the starve medium may be further supplemented with IFN-γ. The starve medium may also be further supplemented with IL-6. In some embodiments of the method, the extracellular vesicles or exosomes have one or more of the following characteristics: (a) enhanced expression of flotillin-1, (b) enhanced expression of CD73, or (c) enhanced expression of IDO. Culturing may be performed by a 2-D culturing method. Culturing may also be performed by a 3-D culturing method.

In some embodiments, the starve medium may be serum-free. In some embodiments, culturing in growth medium occurs for up to about 7 days. In some embodiments, culturing in starve medium may occur for up to about 2 days. Extracellular vesicles or exosomes may be separated by filtration followed by ultracentrifugation.

A culture comprising an increased amount of extracellular vesicles or exosomes is also provided herein. The culture may be produced by a method comprising: (a) culturing stem cells in a growth medium, and (b) then culturing the stem cells in a starve medium supplemented with at least TNF-α.

In some embodiments, the starve medium may be further supplemented with IFN-γ. The increased amount of extracellular vesicles or exosomes as measured by phospholipid quantitation may be at least double the amount produced by a control culture using identical conditions except without supplementing with a cytokine. The method of stimulating mesenchymal stromal cells with inflammatory signals in vitro to generate extracellular vesicles that are more anti-inflammatory in nature (i.e., more potent) may increase the overall production yield of vesicles.

### BRIEF DESCRIPTION OF THE DRAWINGS

FIG. 1 illustrates an exemplary process for MSC preconditioning in two dimensions.

FIG. 2 is a graphical depiction of initial viability studies of MSCs treated with cytokines.

FIG. 3 shows Brightfield images of MSCs treated with various cytokines.

FIG. 4 is a graphical and table characterization of MSCs after cytokine treatment.

FIG. 5 shows a graphical depiction of phospholipid quantitation of extracellular vesicles.

FIG. 6 shows extracellular vesicle protein expression assessed by western blot.

FIG. 7 contains panels of Brightfield images of mesenchymal stem cells treated with various cytokines.

FIG. 8 is a graphical depiction of flow cytometry and total cell yield of mesenchymal stem cells after cytokine treatments.

FIG. 9 shows a graphical depiction of phospholipid quantitation of extracellular vesicles.

FIG. 10 shows extracellular vesicle protein expression assessed by western blot.

FIG. 11 shows Western blot results for exosomal potency markers.

FIG. 12 Western blot results for exosomal potency markers from the first small scale experiment.

### DETAILED DESCRIPTION

### Definitions

Unless otherwise specified, “a” or “an” means “one or more.”

As used herein, the term “expression” means RNA expression and/or protein expression level of one or more genes. In other words, the term “expression” can refer to either RNA expression or protein expression.

As used herein, the terms “isolating” or “isolated,” when used in the context of an extracellular vesicle or exosome isolated from a cell culture or media, refers to an extracellular vesicle or exosome that, by the hand of man, exists apart from its native environment.

As used herein, the term “extracellular vesicles” includes exosomes.

As used herein, the term “exosome” may include any nano-sized vesicle secreted from different cell types that contains any of various biomolecules, such as proteins or nucleic acids. Exosomes may be enveloped in a lipid bilayer membrane, reflecting their origination from endocytic (intracellular) compartments; they typically range from 30-150 nm in diameter, but are not limited to that range. Exosomes are secreted via exocytosis by a variety of cell types, including cancer cells, and subsequently are taken up by target cells, where they communicate information via surface protein signaling as well as through the transfer of lipids, nucleic acids, and other biomolecules. Intercellular communication by exosomes plays a critical role in the regulation of cellular and physiological processes.

As used herein, the term “Flotillin-1” may include a protein that in humans is encoded by the FLOT1 gene. Caveolae are small domains on the inner cell membrane involved in vesicular trafficking and signal transduction. FLOT1 can encode caveolae-associated, integral membrane proteins.

As used herein, “Tumour Necrosis Factor alpha” or “TNF-α”, is an inflammatory cytokine produced by macrophages/monocytes during acute inflammation and is responsible for a diverse range of signaling events within cells, which can lead to necrosis or apoptosis. The protein is also important for resistance to infection and cancers. TNF alpha exerts many of its effects by binding, as a trimer, to either a 55 kDa cell membrane receptor termed TNFR-1 or a 75 kDa cell membrane receptor termed TNFR-2. Both these receptors belong to the TNF receptor superfamily. The defining trait of these receptors is an extra cellular domain comprised of two to six repeats of cysteine rich motifs.

As used herein, interferon gamma (“IFNγ”) is a dimerized soluble cytokine that is a member of the type II class of interferons. IFNγ is secreted by at least T helper cells (e.g., Tₕ1 cells), cytotoxic T cells (T_{C} cells), macrophages, mucosal epithelial cells and NK cells. IFNγ is the only Type II interferon and it is serologically distinct from Type I interferons; it is acid-labile, while the type I variants are acid-stable. IFNγ has antiviral, immunoregulatory, and anti-tumor properties.

As used herein, interleukin 6 (“IL-6” or “IL6”) is an interleukin encoded by the IL6 gene. It is one of a group of related proteins made by leukocytes (white blood cells) and other cells in the body. Interleukin-6 is made mainly by some T lymphocytes. It causes B lymphocytes to make more antibodies and also causes fever by affecting areas of the brain that control body temperature. Interleukin-6 made in the laboratory can be used as a biological response modifier to boost the immune system in cancer therapy. IL-6 may act as both a pro-inflammatory cytokine and an anti-inflammatory myokine. IL-6 is an important mediator of fever and of the acute phase response.

As used herein, the term “population of extracellular vesicles or exosomes” refers to a population of extracellular vesicles or exosomes having a distinct characteristic. The terms “population of extracellular vesicles or exosomes” and “extracellular vesicles or exosomes” can be used interchangeably to refer to a population of extracellular vesicles or exosomes having a distinct characteristic.

As used herein, the term “mesenchymal stromal cell” includes mesenchymal stem cells. Mesenchymal stem cells are cells found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum. Mesenchymal stem cells are capable of differentiating into different germ lines such as mesoderm, endoderm, and ectoderm. Thus, mesenchymal stem cells are capable of differentiating into a large number of cell types including, but not limited to, adipose, osseous, cartilaginous, elastic, muscular, and fibrous connective tissues. The specific lineage-commitment and differentiation pathway entered into by mesenchymal stem cells depends upon various influences, including mechanical influences and/or endogenous bioactive factors, such as growth factors, cytokines, and/or local microenvironmental conditions established by host tissues. Mesenchymal stem cells are thus non-hematopoietic progenitor cells that divide to yield daughter cells that are either stem cells or are precursor cells which in time will irreversibly differentiate to yield a phenotypic cell.

As used herein, “starve medium” means medium that does not contain supplemental proteins, lipids, or growth factors.

### Methods of Treating Inflammatory Diseases and Enhancing EV Yield

A method of treating an inflammatory disease is provided herein. The method may include administering to a subject in need thereof a therapeutically effective amount of isolated extracellular vesicles or exosomes obtained from mesenchymal stem cells pre-conditioned with at least TNF-α, further wherein the isolated extracellular vesicles or exosomes have one or more of the following characteristics: (a) enhanced expression of flotillin-1, (b) enhanced expression of CD73, or (c) enhanced expression of IDO. The mesenchymal stem cells may be derived from bone marrow. In some embodiments, the mesenchymal stem cells may also be further pre-conditioned with IFN-γ or IL-6. In some embodiments, cells are grown in growth media then switched to Alpha-MEM—starve media without serum—and supplemented with cytokines.

The inflammatory disease selected for treatment may include, but is not limited to, bronchopulmonary dysplasia, idiopathic pulmonary fibrosis, and pulmonary hypertension.

A method of isolating extracellular vesicles or exosomes capable of treating an inflammatory disease is also provided herein. The method may include, but is not limited to, the following steps: (a) culturing stem cells in a growth medium, (b) then culturing the stem cells in a starve medium supplemented with at least TNF-α, and (c) separating exosomes or extracellular vesicles from the culture. In some embodiments, the stem cells of the method may be mesenchymal stem cells. In certain preferred embodiments, the mesenchymal stem cells may be derived from bone marrow. In other embodiments, the mesenchymal stem cells may be derived from adipose, skeletal muscle, heart, umbilical cord, or placenta.

In certain embodiments, the starve medium may be further supplemented with IFN-γ. The starve medium may also be further supplemented with IL-6. In some embodiments of the method, the extracellular vesicles or exosomes have one or more of the following characteristics: (a) enhanced expression of flotillin-1, (b) enhanced expression of CD73, or (c) enhanced expression of IDO. Culturing may be performed by a 2-D culturing method. Culturing may also be performed by a 3-D culturing method.

In some embodiments, the starve medium may be serum-free. In some embodiments, culturing in growth medium occurs for up to about seven days. In some embodiments, culturing in growth medium may occur for zero days, one day, two days, three days, four days, five days, six days, or seven days. In some embodiments, culturing in starve medium may occur for up to about two days, including, but not limited to zero days, one day, or two days. Extracellular vesicles or exosomes may be separated by filtration followed by ultracentrifugation.

### Cultures

A culture comprising an increased amount of extracellular vesicles or exosomes is also provided herein. The culture may be produced by a method comprising: (a) culturing stem cells in a growth medium, and (b) then culturing the stem cells in a starve medium supplemented with at least TNF-α.

In some embodiments, the starve medium may be further supplemented with IFN-γ. The increased amount of extracellular vesicles or exosomes as measured by phospholipid quantitation may be at least double the amount produced by a control culture using identical conditions except without supplementing with a cytokine. The method of stimulating mesenchymal stromal cells with inflammatory signals in vitro to generate extracellular vesicles that are more anti-inflammatory in nature (i.e., more potent) may increase the overall production yield of vesicles.

In some embodiments of the culture conditions of the presently claimed methods, the starve media (such as Alpha-MEM) may contain only penstrep (antibiotic) and cytokines. This causes the cells to produce higher amounts of EVs.

### Examples

Since MSCs produce anti-inflammatory factors in response to an inflammatory microenvironment, MSCs can be induced and/or preconditioned by inflammatory cytokines in two-dimensional cell culture to produce more EVs. The methods of the present disclosure allow for both two-dimensional and three-dimensional culture.

In certain embodiments below, the control group was treated the same as the experimental cytokine groups, excluding the addition of cytokines (i.e., in certain embodiments, the control group was grown in growth medium then switched to starve medium without cytokine supplements).

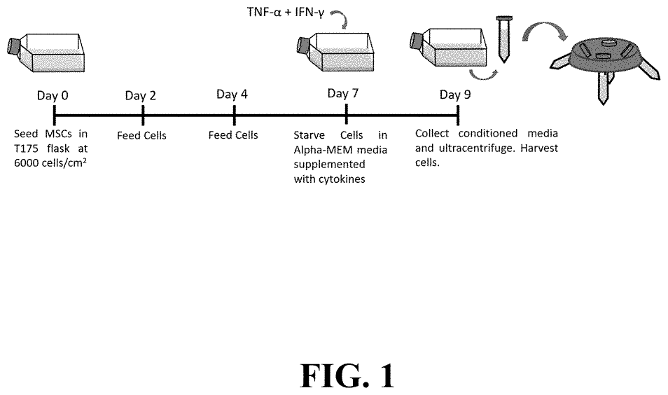

Referring now to FIG. 1, a method is shown in which MSC preconditioning occurs in two dimensions. In 2D cell culture, a variety of cytokines (IL-6, TNF-α, IFN-γ) may be added to media. In some embodiments of the method, cells are cultured for one week. The media may be fed, for example, on Day 2 and Day 4 using growth media. On Day 7, growth media may be removed and replaced with Alpha-MEM (starvation media) supplemented with cytokines. In some embodiments, control groups may be treated the same as the experimental cytokine groups, excluding the addition of cytokines. The control group can be grown in growth medium then switched to starve medium without cytokine supplements.

EV Characterization: After two days, conditioned media may be collected and filtered then ultracentrifuged. The filter may be, for example, a 0.22 μm filter. EV pellets may be resuspended into 1 mL of 1× phosphate buffered saline (PBS) and analyzed by western blot for Flotillin-1, ALIX, IDO, Cleaved Caspase, and CD73. Phospholipid may also be quantified.

MSC Characterization: Cells may also be trypsinized and counted to determine cell yield. In some embodiments, cells may be analyzed by flow cytometry for CD90, CD73, and CD105.

### Results

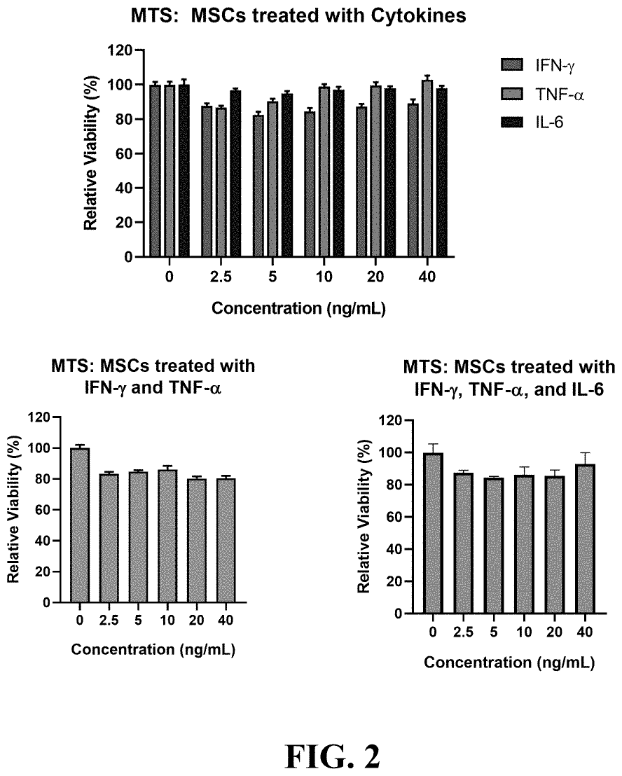

Viability Experiments: In some embodiments, MTS experiments can be done in 96-well plates to test a wide range of cytokine concentrations. Referring now to FIG. 2, initial viability studies of MSCs treated with cytokines are shown. Combination treatments (TNF-α+IFN-γ, and IL-6+TNF-α+IFN-γ) were also tested as well. From these viability experiments, 20 ng/mL was optimized and chosen for small scale experiments.

### 1^{st} Small Scale Treatment: Cytokine Screen

MSCs were first thawed and initially expanded. After expanding the cells, MSCs were trypsinized and seeded at 6000 cells/cm² in a T175 flask where they were cultured for one week. On Day 7, growth media was replaced with starvation media supplemented with 20 ng/mL of IL-6, TNF-α, IFN-γ, TNF-α+IFN-γ, or IL-6+TNF-α+IFN-γ. After two days, conditioned media was processed/analyzed and cells were characterized.

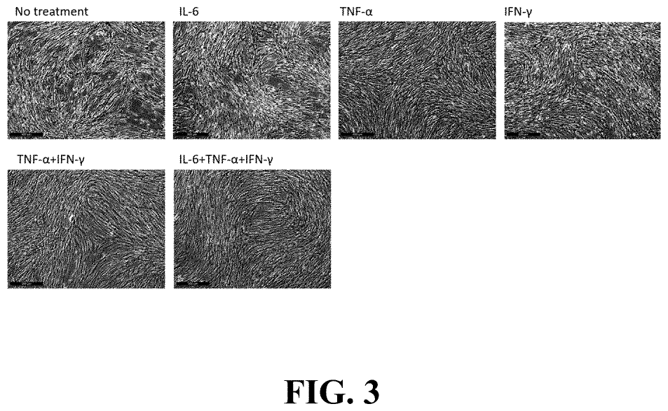

FIG. 3 depicts Brightfield images of MSCs treated with various cytokines. Combination treatments (TNF-α+IFN-γ, and IL-6+TNF-α+IFN-γ) were also tested. From these viability experiments, 20 ng/mL was chosen for the small scale experiments.

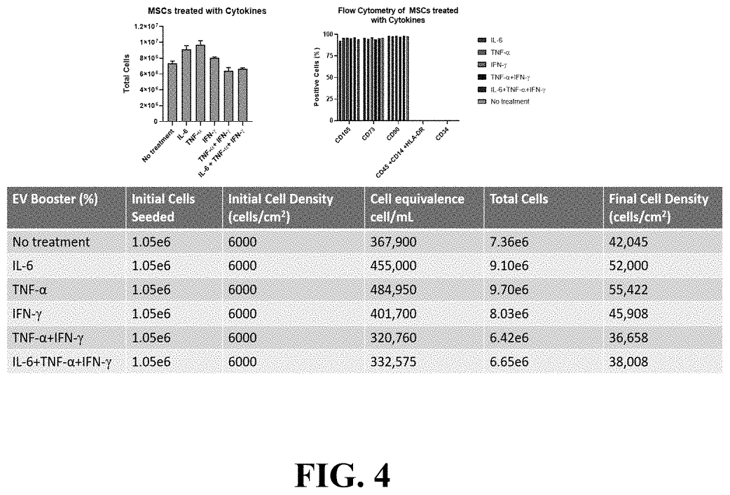

MSC Characterization: Cells were imaged before harvest on Day 9 using 5× magnification. MSCs treated with cytokines showed normal morphology compared to the untreated group. FIG. 4 shows the characterization of MSCs after cytokine treatment. MSCs were harvested in order to determine cell yield. As depicted in FIG. 4, the single cytokine treatment had very little toxicity on cells. When cytokines were combined (TNF-α+IFN-γ, IL-6+TNF-α+IFN-γ), cell yield is shown to slightly decrease. MSCs were fixed using 4% paraformaldehyde then analyzed by flow cytometry. All treatment groups had high expression of CD105, CD73, and CD90.

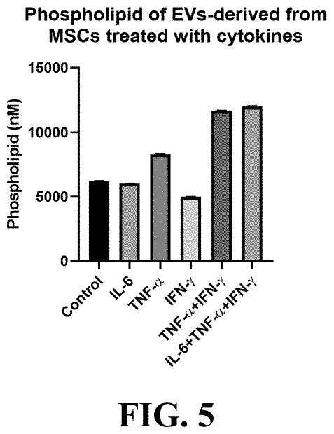

EV Characterization: FIG. 5 illustrates a graphical depiction of phospholipid quantitation of EVs. Phospholipid was measured in the various EV samples derived from MSCs treated with cytokines, conducted by Cambrex. IL-6 and IFN-γ treatments had no effect on phospholipid concentration, similar to results in the non-treated control group. Treatment of MSCs with TNF-α resulted in an increase in phospholipid concentration. However, TNF-α+IFN-γ and IL-6+TNF-α+IFN-γ saw the greatest increase in phospholipid concentration. Since TNF-α+IFN-γ and IL-6+TNF-α+IFN-γ had similar phospholipid concentrations, this suggests that IL-6 does not play a role in the increased secretion of EV. The addition of TNF-α and IFN-γ (dual treatment) is synergistic and this unexpected result shows that both are needed to increase EV secretion and phospholipid content.

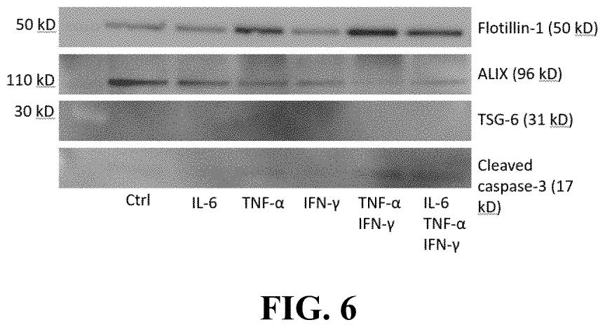

Referring now to FIG. 6, results depicting EV protein expression as assessed by western blot are shown. All EV samples expressed Flotillin-1 and ALIX. However, results also show higher expression of Flotillin-1 in the TNF-α+IFN-γ and IL-6+TNF-α+IFN-γ conditions. Cleaved caspase-3 was also detected in the combination treatments as well, suggesting that apoptotic bodies are present.

### 2^{nd} Small Scale Treatment: Varying Concentration of TNF-α & IFN-γ

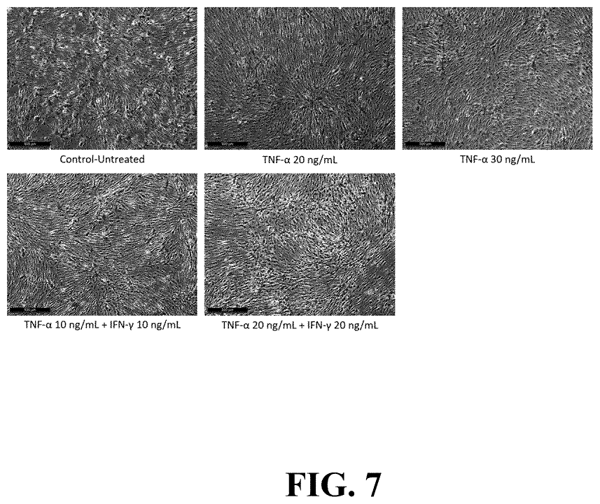

MSC Characterization: Referring now to FIG. 7, Brightfield images of MSCs treated with various cytokines are shown. As depicted in the images, all cells maintained normal morphology. In particular, MSCs treated with TNF-α 20 ng/mL & IFN-γ 20 ng/mL were more elongated and thinner, but still maintained normal morphology.

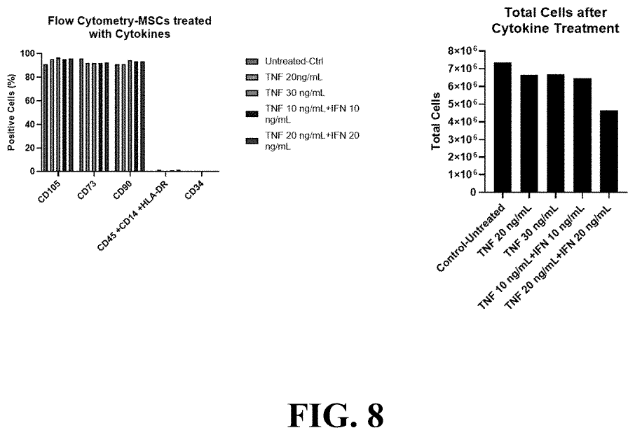

FIG. 8 graphically conveys the flow cytometry and total cell yield of MSCs after cytokine treatments. On day 9, cells were harvested and characterized by flow cytometry. All MSCs had similar expression of CD105, CD73, and CD90 compared to the untreated (control) condition. Total cell yield was also determined. Cell yield decreased slightly and were similar between TNF-α at 20 ng/ mL and 30 ng/mL as well as TNF-α 10 ng/mL+IFN-γ 10 ng/mL. TNF-α 20 ng/mL+IFN-γ 20 ng/mL caused the highest reduction in cell yield, which may have been due to lack of sufficient humidity in the incubator.

### EV Characterization

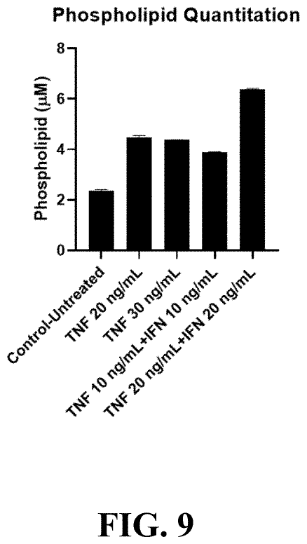

FIG. 9 shows phospholipid Quantitation of EVs from the second small scale treatment. After processing the EVs on day 9, phospholipid concentration was measured. TNF-α at 20 ng/ mL and 30 ng/mL as well as TNF-α 10 ng/mL+IFN-γ 10 ng/mL caused phospholipid to increase. However, the TNF-α 20 ng/mL+IFN-γ 20 ng/mL condition experienced the highest increase in phospholipid concentration (phospholipid analysis was performed in house at United Therapeutics).

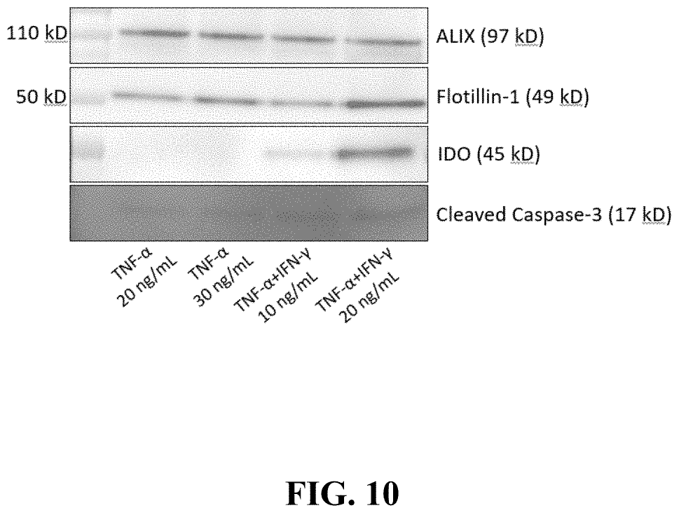

Referring now to FIG. 10, EV protein expression as assessed by western blot analysis is shown. All EV preparations contained ALIX and Flotillin-1. However, the highest expression of Flotillin-1 occurs in the TNF-α 20 ng/mL+IFN-γ 20 ng/mL condition. IDO expression is only seen in the dual treatment groups, with TNF-α 20 ng/mL+IFN-γ 20 ng/mL EVs having the highest expression of IDO. Cleaved caspase-3 is present in all conditions.

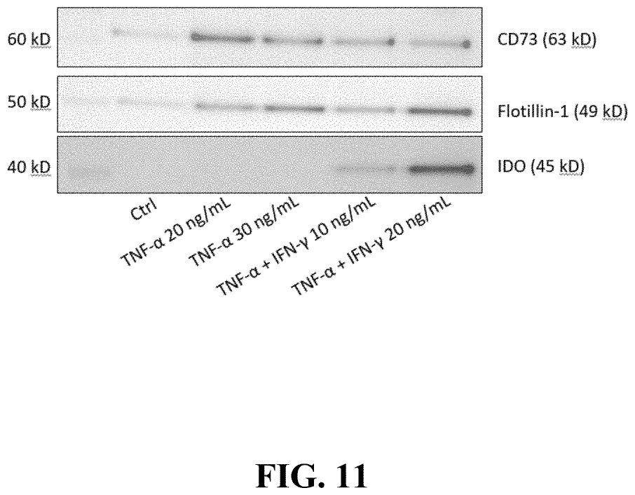

FIG. 11 shows Western blot analysis for three exosomal potency markers, CD 73 (63 kD), Flotillin-1 (49 kD), and IDO (45 kD). The western blot was repeated again to include the untreated control next to the experimental samples. Flotillin-1/IDO expression remained the same. Of noteworthy significance, the EVs from the untreated group also did not have IDO expression, which is seen as a potency marker. An additional protein marker that was tested was CD73, which was present in all EV groups.

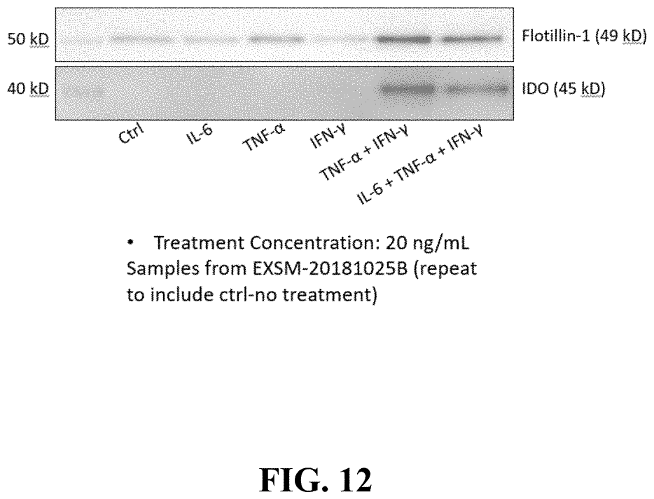

As depicted in FIG. 12, Western blot is shown for exosomal potency markers from the 1^{st} small scale experiment. EVs from the 1^{st} small scale experiment were tested one more time. It was hypothesized that IFN-γ could be contributing to exosomal IDO expression. However, IDO expression was not detected from EVs derived from the IFN-γ treatment group. This data demonstrates that both TNF-α 20 ng/mL+IFN-γ 20 ng/mL are essential for preconditioning MSCs to secrete more EVs, and particularly to secrete EVs that express potency markers.

### 3-D Culturing Method

In some embodiments, cells may be grown in a three-dimensional (3D) format and for the same or similar culture times as those discussed above for two-dimensional format, incorporating the starvation period. The methods can be used to treat a variety of inflammatory diseases, including but not limited to, bronchopulmonary dysplasia, idiopathic pulmonary fibrosis, and/or other inflammatory diseases.

### Additional Methods

Alternative ways to practice the invention include using the cytokines in various concentrations/combinations. In some embodiments, the cytokines used to treat the culture include at least one of, or any combination of, IL-6, TNF-α, or IFN-γ. In certain embodiments, the concentration of cytokines is anywhere from 1-100 ng/mL, or any value therebetween. Other components that could be used to stress MSCs to produce more EVs include other types of inflammatory cytokines, anti-inflammatory cytokines, growth factors, hormones, or TLR-agonists.

Substitute cellular stressor materials may include, for example:
Inflammatory cytokines: IL-1β, IL-1α, IL-8
Anti-inflammatory cytokines: IL-10
Growth factors: FGF, TGF-beta, TGF-alpha, IGF-1, BMP-2
Hormones: Oxytocin, adiponectin, glucocorticoid
TLR-agonists: poly(I:C)
Hypoxia: 1-5% O₂

Another alternative method that can be used is to vary the cell line. Preconditioning can be done on MSCs from other tissue sources or on immortalized MSCs. In certain preferred embodiments, bone-marrow derived MSCs may be used. However, the methods of the present disclosure may be expanded to MSCs derived from, but not limited to, wharton's jelly, umbilical cord, amniotic fluid, cord blood, peripheral blood, placenta, or adipose tissue.

In some embodiments, bioreactors are able to produce increased cell yields compared to conventional 2D culture. Types of bioreactors may include, but are not limited to, stir-tank, packed bed, hollow fiber, and PBS systems. With increased cell yield, more EVs are able to be secreted, however, they may not necessarily be more potent than in 2D culture. By combining the cytokine preconditioning in bioreactors, EV yield and potentially potency can both be increased. With the increase in EV yield, this method may prove to be cost-effective and allow for fewer production runs.

### REFERENCES

1. Domenis R, Cifù A, Quaglia S, Pistis C, Moretti M, Vicario A, Parodi P C, Fabris M, Niazi K R, Soon-Shiong P, Curcio F. Pro inflammatory stimuli enhance the immunosuppressive functions of adipose mesenchymal stem cells-derived exosomes.Sci Rep. 2018 Sep. 6; 8(1):13325. doi: 10.1038/s41598-018-31707-9. PubMed PMID: 30190615; PubMed Central PMCID: PMC6127134.
2. Ferreira J R, Teixeira G Q, Santos S G, Barbosa M A, Almeida-Porada G, Gonsalves RM.Mesenchymal Stromal Cell Secretome: Influencing Therapeutic Potential by Cellular Pre-conditioning. Front Immunol. 2018; 9:2837. doi: 10.3389/fimmu.2018.02837. eCollection 2018. Review. PubMed PMID: 30564236; PubMed Central PMCID: PMC6288292.
3. Hemeda H, Jakob M, Ludwig A K, Giebel B, Lang S, Brandau S. Interferon-gamma and tumor necrosis factor-alpha differentially affect cytokine expression and migration properties of mesenchymal stem cells. Stem Cells Dev. 2010 May; 19(5):693-706. doi: 10.1089/scd.2009.0365. PubMed PMID: 20067407.
4. Chen H, Min X H, Wang Q Y, Leung F W, Shi L, Zhou Y, Yu T, Wang C M, An G, Sha W H, Chen Q K. Pre-activation of mesenchymal stem cells with TNF-α, IL-1β and nitric oxide enhances its paracrine effects on radiation-induced intestinal injury. Sci Rep. 2015 Mar. 3;5:8718. doi: 10.1038/srep08718. PubMed PMID: 25732721; PubMed Central PMCID: PMC4346809.
5. Hu C, Li L. Preconditioning influences mesenchymal stem cell properties in vitro and in vivo. J Cell Mol Med. 2018 March; 22(3):1428-1442. doi: 10.1111/jcmm.13492. Epub 2018 Feb. 1. Review. PubMed PMID: 29392844; PubMed Central PMCID: PMC5824372.
6. Saparov A, Ogay V, Nurgozhin T, Jumabay M, Chen W C. Preconditioning of Human Mesenchymal Stem Cells to Enhance Their Regulation of the Immune Response. Stem Cells Int. 2016;2016:3924858. doi: 10.1155/2016/3924858. Epub 2016 Oct. 16. Review. PubMed PMID: 27822228; PubMed Central PMCID: PMC5086389.
7. Cunningham C J, Redondo-Castro E, Allan S M. The therapeutic potential of the mesenchymal stem cell secretome in ischaemic stroke. J Cereb Blood Flow Metab. 2018 August; 38(8):1276-1292. doi: 10.1177/0271678X18776802. Epub 2018 May 17. PubMed PMID: 29768965; PubMed Central PMCID: PMC6077926.
8. Lin T, Pajarinen J, Nabeshima A, Lu L, Nathan K, Jamsen E, Yao Z, Goodman S B. Preconditioning of murine mesenchymal stem cells synergistically enhanced immunomodulation and osteogenesis. Stem Cell Res Ther. 2017 Dec. 6; 8(1):277. doi: 10.1186/s13287-017-0730-z. PubMed PMID: 29212557; PubMed Central PMCID: PMC5719931.
9. Redondo-Castro E, Cunningham C, Miller J, Martuscelli L, Aoulad-Ali S, Rothwell N J, Kielty C M, Allan S M, Pinteaux E. Interleukin-1 primes human mesenchymal stem cells towards an anti-inflammatory and pro-trophic phenotype in vitro. Stem Cell Res Ther. 2017 Apr. 17; 8(1):79. doi: 10.1186/s13287-017-0531-4. PubMed PMID: 28412968; PubMed Central PMCID: PMC5393041.
10. Ma H C, Wang X, Wu M N, Zhao X, Yuan W, Shi X L. Interleukin-10 Contributes to Therapeutic Effect of Mesenchymal Stem Cells for Acute Liver Failure via Signal Transducer and Activator of Transcription 3 Signaling Pathway. Chin Med J (Engl). 2016 Apr 20; 129(8):967-75. doi: 10.4103/0366-6999.179794. PubMed PMID: 27064043; PubMed Central PMCID: PMC4831533.
11. Zeuner M T, Patel K, Denecke B, Giebel B, Widera D. Paracrine effects of TLR4-polarised mesenchymal stromal cells are mediated by extracellular vesicles. J Transl Med. 2016 Feb. 2; 14:34. doi: 10.1186/s12967-016-0794-z. PubMed PMID: 26838370; PubMed Central PMCID: PMC4735950.
12. Xu C, Yu P, Han X, Du L, Gan J, Wang Y, Shi Y. TGF-β promotes immune responses in the presence of mesenchymal stem cells. J Immunol. 2014 Jan. 1; 192(1):103-9. doi: 10.4049/jimmunol.1302164. Epub 2013 Nov. 29. PubMed PMID: 24293629.
13. Ankrum J A, Dastidar R G, Ong J F, Leby O, Karp J M. Performance-enhanced mesenchymal stem cells via intracellular delivery of steroids. Sci Rep. 2014 Apr. 10; 4:4645.

1. A method of treating an inflammatory disease, comprising administering to a subject in need thereof a therapeutically effective amount of isolated extracellular vesicles or exosomes obtained from mesenchymal stem cells pre-conditioned with at least TNF-α, wherein the isolated extracellular vesicles or exosomes have one or more of the following characteristics:
(a) enhanced expression of flotillin-1,
(b) enhanced expression of CD73, or
(c) enhanced expression of IDO.

2. The method of claim 1, wherein the mesenchymal stem cells are derived from bone marrow.

3. The method of claim 1, wherein the mesenchymal stem cells are further pre-conditioned with IFN-γ.

4. The method of claim 3, wherein the mesenchymal stem cells are further pre-conditioned with IL-6.

5. The method of claim 1, wherein inflammatory disease is selected from the group consisting of bronchopulmonary dysplasia, idiopathic pulmonary fibrosis, and pulmonary hypertension.

6. A method of isolating extracellular vesicles or exosomes capable of treating an inflammatory disease, comprising:
(a) culturing stem cells in a growth medium,
(b) then culturing the stem cells in a starve medium supplemented with at least TNF-α, and
(c) separating exosomes or extracellular vesicles from the culture.

7. The method of claim 6, wherein the stem cells are mesenchymal stem cells.

8. The method of claim 7, wherein the mesenchymal stem cells are derived from bone marrow.

9. The method of claim 6, wherein the starve medium is further supplemented with IFN-γ.

10. The method of claim 9, wherein the starve medium is further supplemented with IL-6.

11. The method of claim 6, wherein the extracellular vesicles or exosomes have one or more of the following characteristics:
(a) enhanced expression of flotillin-1,
(b) enhanced expression of CD73, or
(c) enhanced expression of IDO.

12. The method of claim 6, wherein the culturing is performed by a 2-D culturing method.

13. The method of claim 6, wherein the culturing is performed by a 3-D culturing method.

14. The method of claim 6, wherein the starve medium is serum-free.

15. The method of claim 6, wherein culturing in growth medium occurs for up to about 7 days.

16. The method of claim 6, wherein culturing in starve medium occurs for up to about 2 days.

17. The method of claim 6, wherein the extracellular vesicles or exosomes are separated by filtration followed by ultracentrifugation.

18. A culture comprising an increased amount of extracellular vesicles or exosomes, which is produced by a method comprising:
(a) culturing stem cells in a growth medium, and
(b) then culturing the stem cells in a starve medium supplemented with at least TNF-α.

19. The culture of claim 18, wherein the starve medium is further supplemented with IFN-γ.

20. The culture of claim 19, wherein the increased amount of extracellular vesicles or exosomes as measured by phospholipid quantitation is at least double the amount produced by a control culture using identical conditions except without supplementing with a cytokine.
